(19) United States
(12) Patent Application Publication
(10) Pub. No.: US 2017/0102348 A1
(43) Pub. Date: Apr. 13, 2017

(54) ELECTROCHEMICAL BIOSENSOR FOR METABOLIC DISEASE OF CATTLE

(71) Applicant: University of Guelph, Guelph (CA)

(72) Inventors: Suresh Neethirajan, Guelph (CA); Murugan Veerapandian, Guelph (CA)

(21) Appl. No.: 15/288,171

(22) Filed: Oct. 7, 2016

Related U.S. Application Data
(60) Provisional application No. 62/239,474, filed on Oct. 9, 2015.

(51) Int. Cl. G01N 27/327; C12Q 1/00

(52) U.S. Cl. CPC G01N 27/3272; C12Q 1/005

(57) ABSTRACT

The present application relates to biosensors and methods for detecting and/or quantifying a target substrate. The biosensors comprise an electrode with a surface layer of tris-ruthenium bipyridine²⁺ complex modified graphene oxide and at least one enzyme linked to the surface layer.

### RELATED APPLICATION

The present application claims the benefit of priority of U.S. Provisional Application Ser. No. 62/239,474 filed Oct. 9, 2015 the entire contents of which are hereby incorporated by reference.

### FIELD

The present application relates to the electrochemical detection of biomarkers and more specifically to sensors and methods for detecting and/or quantifying biomarkers in a liquid sample using surface-modified electrodes.

### BACKGROUND

Incidence of Negative Energy Balance (NEB) in dairy cattle caused by increased energy demands at periparturient period is a serious illness that often affects livestock (Jorjong et al., 2014). Circulating Non-Esterified Fatty Acid (NEFA) and β-hydroxybutyrate (BHBA) levels are a good indicator of NEB. During NEB, adipose fat is mobilized as NEFA and transported to the liver to be oxidized or re-esterified into triglycerides. Excessive amounts of NEFA removed by the liver of the dairy cow along with carnitine palmitoyaltransferase-1 activity results in ketogenesis, and thus elevate BHBA levels (Ospina et al., 2010a). Elevated NEFA and BHBA in blood plasma are detrimental for dairy cows (Garverick et al., 2013). Clinical disease associated with periparturient conditions include fatty liver, ketosis, displaced abomasum, metritis and retained placenta (Ospina et al., 2010a, 2010b). Ketosis (clinical and subclinical) occurs due to NEB in dairy cows. The presence of excess ketone bodies without clinical signs is defined as Subclinical Ketosis (SCK). Early diagnosis of SCK is vital to optimize herd management for preventing outbreaks of clinical disease (Nielen et al., 1994). Further, dairy cows with SCK tend to have reduced milk production relative to those with normal ketone body concentrations. It is clear that the determination of NEFA and BHBA are of significant clinical value for dairy health management, and of economical value for livestock producers.

Diagnosis of SCK may be performed by measuring blood concentrations of NEFA and/or BHBA. Threshold blood NEFA concentrations at pre-partum and post-partum are ≧0.3 and ≧0.6 mEq/L, respectively, whereas a BHBA concentration range from 1.2 to 2.9 mM is associated with SCK (Ospina et al., 2010a). Constant monitoring of NEFA and BHBA is therefore an integral part of managing the health of livestock and in particular dairy cows.

Typically, due to a lack of on-farm diagnostic tests for NEFA and BHBA, blood samples are collected and sent to the off-site laboratories for further testing. NEFA is often quantified using high performance liquid chromatography (HPLC), gas chromatography/mass spectrometry (GC/MS), and liquid chromatography/mass spectrometry (LC/MS) (Miska et al., 2004). Matrix-assisted laser desorption ionization/mass spectrometry (MALDI/MS) has received widespread attention in fatty acids analysis (Yang and Fujino 2014). Quantification of free fatty acids in human serum has also been demonstrated using HPLC with fluorescence (Nishikiori et al., 2014), and a chip-based direct-infusion nanoelectrospray ionization source coupled to Fourier transform ion cyclotron resonance MS (Zhang et al., 2014). However, these techniques are expensive, time consuming, require specialized technical operating personnel, and further rely on large laboratory equipment.

In-vitro enzymatic colorimetric assay kits are available from Roche and Wako diagnostics for the detection of oleic acid and palmitic acid, the major NEFA components in serum. However, these methods involve three steps utilizing two enzymes (acyl-CoA synthetase (ACS) and acyl-CoA oxidase (ACOD)), and additional reagents for generating pigmented products, which are then quantified by UV-spectrometer at a specific wavelength.

Likewise, blood ketone bodies are typically determined indirectly using chromatographic, isotopic and spectrophotometric methods (Fang et al. 2008, Khorsand et al., 2013). The electrochemical detection of BHBA has been described using the enzyme β-hydroxybutyrate dehydrogenase (HBDH) supported with pyridine coenzyme [NAD(P)+] as the electron acceptor (Forrow et al., 2005, Kwan et al., 2006, Li et al., 2005). However, interference by electroactive species and their influence on enzyme activity are drawbacks of such indirect biosensing techniques (Forrow et al., 2005, Kwan et al., 2006). Direct electrochemical detection of NADH oxidation based on iridium-carbon (Fang et al., 2008) and functionalized single walled carbon nanotubes (SWCNTs) (Khorsand et al., 2013) modified electrode supported with cofactor NAD+ has been demonstrated. However, these techniques have predominantly focused on the diagnosis of diabetic ketoacidosis in humans and may not be suitable for other species. For example, cows have 11 major blood groups (A, B, C, F, J, L, M, R, S, T and Z) unlike the 4 groups in humans, owing to the expression of different antigens, which make it complex and inaccurate to determine BHBA in cows using a human medicine ketosis detector (Antalikov et al., (2007)).

There remains a need for simple, sensitive and commercially feasible sensors for the detection of animal biomarkers as NEFA and BHBA.

### SUMMARY

In one aspect, the disclosure provides an electrochemical sensor based on ruthenium bispyridine complex modified graphene oxide ([Ru(bpy)₃]²⁺-GO). As shown in Example 1, [Ru(bpy)₃]²⁺-GO electrodes exhibit superior and durable redox properties compared to pristine carbon and GO electrodes. The sensor may be used to detect and/or quantify a target biomarker. One or more enzymes may be immobilized onto the surface of the electrode, which catalyze the production of redox active species from the target biomarker. Electrochemical techniques may then be used to detect the amperometric response to the presence of redox active species in a sample.

For example, in one embodiment the sensor may be for the detection of β-hydroxybutyrate (BHBA) and an enzyme with β-hydroxybutyrate dehydrogenase activity is linked to the surface of the electrode. In another embodiment, the sensor may be for the detection of non-esterified fatty acids (NEFA) and an enzyme with lipoxygenase activity is linked to the surface of the electrode. As shown in the Examples, β-hydroxybutyrate dehydrogenase and lipoxygenase enzymes retain their catalytic ability upon immobilization to [Ru(bpy)₃]²⁺-GO electrodes and exhibit changes to amperometric signals upon interaction with relevant analyte concentrations.

The [Ru(bpy)₃]²⁺-GO electrodes described herein may therefore be used as biosensor platform for deployable, rapid and user-friendly devices for the detection and/or quantification of analytes such as enzyme substrates.

In one embodiment, the [Ru(bpy)₃]²⁺-GO electrodes described may be used for detecting levels of the biomarkers NEFA and/or BHBA. Optionally, the biosensors and methods described herein are for the detection, diagnosis and/or management of metabolic diseases such as negative energy balance or clinical or subclinical ketosis.

Accordingly, in one embodiment there is provided an electrochemical biosensor comprising an electrode comprising a surface layer of tris-ruthenium bipyridine²⁺ complex modified graphene oxide. In one embodiment, the biosensor further comprises at least one enzyme linked to the surface layer. In one embodiment, the enzyme is covalently linked to the surface layer.

In one embodiment, the electrode is a screen printed electrode. In one embodiment, the tris-ruthenium bipyridine²⁺ complex modified graphene oxide is drop cast onto the surface of the electrode.

In one embodiment, the enzyme linked to the surface layer has lipoxygenase activity and catalyzes the oxidation of polyunsaturated fatty acids to form a peroxide of the acid. In one embodiment, the enzyme is lipoxygenase (EC 1.13.11).

In another embodiment, the enzyme has β-hydroxybutyrate dehydrogenase activity and catalyzes the production of acetoacetate in the presence of nicotinamide adenine dinucleotide (NAD+). In one embodiment, the enzyme is β-hydroxybutyrate dehydrogenase (EC 1.1.1.30). In one embodiment, the biosensor further comprises nicotinamide adenine dinucleotide (NAD+) linked to the surface layer.

Optionally, the biosensors may include one or more [Ru(bpy)₃]²⁺-GO electrodes as described herein. For example, in one embodiment the biosensor comprises:

a first electrode comprising a surface layer of tris-ruthenium bipyridine²⁺ complex modified graphene oxide and a first enzyme linked to the surface layer of the first electrode, and

a second electrode comprising a surface layer of tris-ruthenium bipyridine²⁺ complex modified graphene oxide and a second enzyme linked to the surface layer of the second electrode.

In one embodiment, the first electrode and the second electrode are on separate substrates. In another embodiment, the first electrode and second electrode are on the same substrate.

In one embodiment, the biosensors described herein include at least one counter electrode and at least one reference electrode optionally on the same substrate as the [Ru(bpy)₃]²⁺-GO electrode or on different substrates.

The biosensors described herein may be incorporated into various devices for the detection and/or quantification of target analytes. In one embodiment, the biosensors described herein are for detecting and/or quantifying a target substrate in a fluid sample. In one embodiment the biosensor is a hand-held system. In another embodiment the biosensor is part of an in-line system, such a fluid management system for collecting and or processing milk.

In another aspect, there is provided a method for detecting and/or quantifying the level of a target substrate in a sample. In one embodiment, the method comprises:

a) applying at least one voltage across the electrode of a biosensor as described herein and a counter electrode in the presence of the sample, and

b) detecting at least one current value in response to the at least one voltage.

In one embodiment, the magnitude of the current value is proportional to the level of the target substrate in the sample. In one embodiment, the method further comprises comparing at least one current value to at least one standard current value, wherein the standard current value is representative of the current for a known concentration of the target substrate in the sample.

In one embodiment, the target substrate is non-esterified fatty acids (NEFA) and the biosensor comprises lipoxygenase linked to the surface layer. Alternatively or in addition, the target substrate is β-hydroxybutyrate (BHBA) and the biosensor comprises β-hydroxybutyrate dehydrogenase and NAD+ linked to the surface layer.

In one embodiment, the sample is a biological fluid from a subject. In one embodiment, the biological fluid is blood, blood plasma or milk. In one embodiment, the subject is an animal, optionally a livestock animal. In one embodiment, the livestock animal is a bovid, optionally a dairy cow.

In one embodiment, the biosensors and methods described herein are for the detection of metabolic disease. In one embodiment, the metabolic disease is negative energy balance (NEB). In one embodiment, the metabolic disease is clinical or subclinical ketosis.

Other features and advantages of the present application will become apparent from the following detailed description. It should be understood, however, that the detailed description and the specific examples while indicating embodiments of the application are given by way of illustration only and the scope of the claims should not be limited by the embodiments set forth in the examples, but should be given the broadest interpretation consistent with the description as a whole.

### BRIEF DESCRIPTION OF THE DRAWINGS

The present application will now be described in greater detail with reference to the drawings in which:

FIG. 1 shows (A) UV-vis absorbance spectra, (B) Raman spectra (asterisk denotes the C—N of bipyridyl groups), (C) XPS spectra of C1s, (D) TEM and (E) SEM images of (i) pristine GO and (ii) [Ru(bpy)₃]²⁺-GO.

FIG. 2 shows the production of enzyme-modified Rubpy-GO electrodes. (A) Enzyme SLO modification on Rubpy-GO electrode and its enzymatic reaction with NEFA sample. (B) Modification of HBDH on Rubpy-GO/NAD+ electrode surface and its reaction with BHBA sample. Rubpy denotes [Ru(bpy)₃]²⁺. WE represents the working electrode.

FIG. 3 (A) CV of bare CSPE, GO and [Ru(bpy)₃]²⁺-GO electrode measured under PBS (pH 7.4) at a scan rate of 20 mV/s. (B) CVs of [Ru(bpy)₃]²⁺-GO electrode at various scan rates from 10 to 100 mV/s (inset shows the respective peak current plots vs square root of scan rate. (C) CV of [Ru(bpy)₃]²⁺-GO/SLO electrode under PBS buffer (pH 7.4) at a scan rate of 20 mV/s, in the absence and presence of NEFA (0.4 mM). (D) Calibration curve fit representing the amperometric current of Ru(bpy)₃]²⁺-GO/SLO against various concentrations of NEFA in PBS (pH 7.4). Each data point indicates the average of two independent measurements at different electrodes, and error bars indicate the standard deviation of the mean. (E) Amperometric histogram of [Ru(bpy)₃]²⁺-GO/SLO electrode measured in the presence of NEFA (0.4 mM) and interferents (1.2 mM). (F) Amperometric response from four different serum samples measured at [Ru(bpy)₃]²⁺-GO/SLO electrodes. All the amperometric measurements were recorded at an applied bias of +0.17 V.

FIG. 4 shows the electrochemical enzymatic reaction sequence for (A) NEFA and (B) BHBA on the surface of [Ru(bpy)₃]²⁺-GO and [Ru(bpy)₃]²⁺-GO/NAD+ electrodes, respectively.

FIG. 5 shows CV of [Ru(bpy)₃]²⁺-GO/NAD+ electrode measured under PBS (pH 7.4) at a scan rate of 20 mV/s. (B) CVs of [Ru(bpy)3]²⁺-GO/NAD+ electrode at various scan rates from 10 to 100 mV/s (inset shows the respective peak current plots vs square root of scan rate, (C) CV of [Ru(bpy)₃]²⁺-GO/NAD+/HBDH electrode under PBS buffer (pH 7.4) at a scan rate of 50 mV/s, in absence (a) and presence (b) of BHBA (0.4 mM). (D) Calibration curve fit representing the amperometric current of [Ru(bpy)₃]²⁺-GO/NAD+/HBDH against various concentrations of BHBA in PBS (pH 7.4). Each data point indicates the average of two independent measurements at different electrodes, and error bars indicate the standard deviation of the mean. (E) Amperometric histogram of [Ru(bpy)₃]²⁺-GO/NAD+/HBDH electrode measured in the presence of BHBA (0.2 mM) and interferents (1.2 mM). (F) Amperometric response from four different serum samples measured at [Ru(bpy)₃]²⁺-GO/NAD+/HBDH electrodes. All the amperometric measurements were recorded at an applied bias of +0.06 V.

FIG. 6 shows (A) Prototype dual electrode design screen-printed on a single chip integrated with reference (RE) and counter (CE) electrodes, respectively. (B) Two working electrode substrates modified selectively with different enzymes targeting NEFA/BHBA and model voltammetry signal.

FIG. 7 shows an SEM image of GO nanosheets with identified elemental mapping of uniformly distributed C—K and O—K.

FIG. 8 shows SEM image of [Ru(bpy)₃]²⁺-GO nanosheets with identified elemental mapping of uniformly distributed C—K, O—K and Ru-L.

FIG. 9 shows CVs of bare carbon screen printed electrode under PBS buffer (pH 7.4), at a scan rate of 20 mV/s, in absence and presence of NEFA sample (0.4 mM).

FIG. 10 shows CVs of GO-modified carbon screen printed electrode under PBS buffer (pH 7.4), at a scan rate of 20 mV/s, in absence and presence of NEFA sample (0.4 mM).

FIG. 11 shows (A) LSV response of [Ru(bpy)₃]²⁺-GO/SLO electrode against various NEFA concentrations in PBS buffer (pH 7.4), at a scan rate of 20 mV/s. (B) Linear response of steady state current (at +0.17 V) with NEFA concentration. Error bars indicate the standard deviation of the mean measured from two different electrodes. Normalized current (/N) denotes the change of current measured from electrodes under NEFA and PBS (/N=/NEFA−/PBS).

FIG. 12 shows the amperometric response of [Ru(bpy)₃]²⁺-GO/SLO electrode (black, square plot) and WAKO kit based colorimetric response (orange, circle plot) against various concentrations of dairy cow clinical serum samples, respectively.

### DETAILED DESCRIPTION

### I. Biosensors and Devices

In one aspect of the disclosure there is provided a biosensor comprising an electrode suitable for the electrochemical detection of a target analyte. The inventors have determined that ruthenium bispyridine complex modified graphene oxide ([Ru(byp)₃]²⁺-GO) is a particularly advantageous material for use on the surface of electrodes. As shown in the Examples, [Ru(byp)₃]²⁺-GO electrodes exhibit superior redox properties relative to carbon or GO electrodes.

Furthermore, [Ru(byp)₃]²⁺-GO surface modified electrodes can be further modified to link one or more enzymes to the electrode surface and still maintain enzymatic activity. In one embodiment, the electrode surface is modified with an enzyme that selectively binds to a target substrate and produces redox active species from the target substrate resulting in detectable changes to an amperometric signal.

Accordingly, in one embodiment, there is provided an electrochemical biosensor comprising:

an electrode comprising a surface layer of tris-ruthenium bipyridine²⁺ complex modified graphene oxide; and

at least one enzyme linked to the surface layer.

The electrode may be made of any material suitable for depositing a surface layer of tris-ruthenium bipyridine²⁺ complex modified graphene oxide. For example, in one embodiment the electrode is made from a conductive material such as carbon, gold, silver, copper, aluminum, graphite, brass, platinum, palladium or titanium.

In one embodiment, the electrode is a screen printed electrode. In one embodiment, the electrode is a printed carbon electrode. The electrode may optionally be on a substrate material such as, but no limited to, a vinyl web, polyethylene terepthalate, glass or paper.

Various methods known in the art may be used to apply a layer of tris-ruthenium bipyridine²⁺ complex modified graphene oxide to the surface of the electrode. For example, in one embodiment tris-ruthenium bipyridine²⁺ complex modified graphene oxide is drop cast onto the surface of the electrode. Optionally, more than one layer of casting may be applied to the electrode to ensure a uniform application of tris-ruthenium bipyridine²⁺ complex modified graphene oxide. Other methods for coating tris-ruthenium bipyridine²⁺ complex modified graphene oxide on the electrode include drop coating, vacuum filtration deposition method, electrochemical reduction deposition, or plasma assisted electrode deposition.

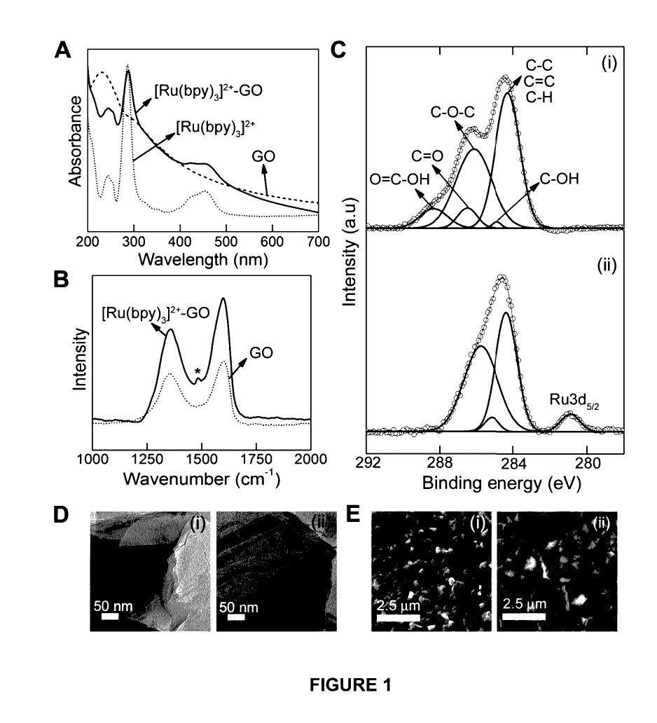

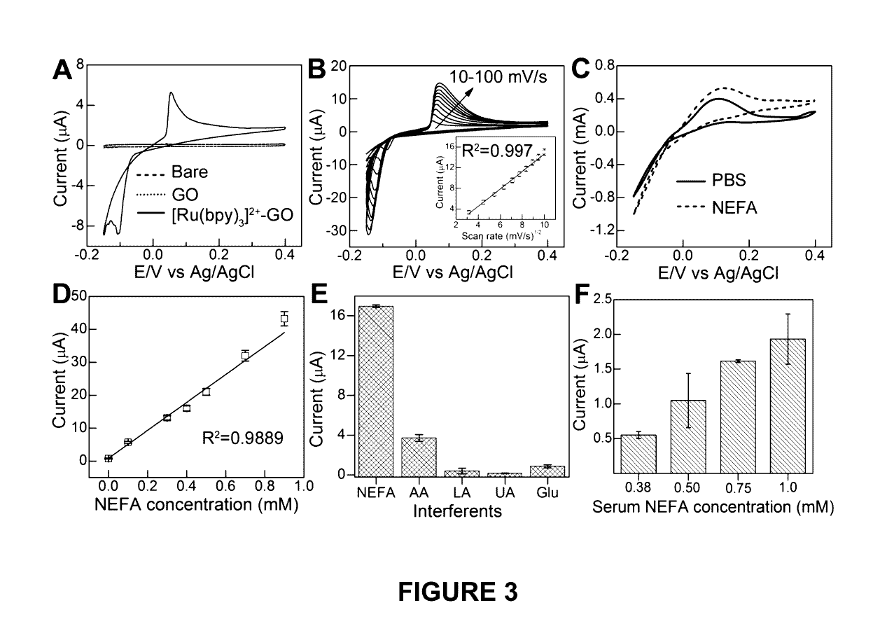

As shown in FIGS. 1 and 3, ruthenium bipyridine complex modified graphene oxide has a characteristic sheetlike structure and exhibits particular spectroscopic and electrochemical properties. For example, in one embodiment Raman spectra of ruthenium bipyridine complex modified graphene oxide nanosheets exhibit intrinsic D and G-bands as well as a band at 1481 cm⁻¹ attributed to C—N stretching vibrations of bipyridyl groups. The ruthenium bipyridine complex modified graphene oxide electrodes described herein exhibit a number of advantageous characteristics for the electrochemical detection of analytes. For example, ruthenium bipyridine complex modified graphene oxide electrodes are durable and effective at transducing the electron transfer process at the sensor interface. Furthermore, due to its matrix-like structure with abundant chemical groups, ruthenium bipyridine complex modified graphene oxide readily adheres to the surface of electrodes. In addition, the redox response of ruthenium bipyridine complex modified graphene oxide is superior to other materials. Ruthenium bipyridine complex modified graphene oxide is also cost-effective and presents a scalable synthesis for use in the commercial production of biosensors.

In another aspect, at least one enzyme is linked to the surface layer of tris-ruthenium bipyridine²⁺ complex modified graphene oxide. In one embodiment, the enzyme is covalently linked to the surface layer. Optionally, the enzyme may be directly linked to the surface layer or may be linked through a linker, such as a polypeptide chain. In one embodiment, the enzyme may be linked to the surface layer by physicosorption through drop casting of the enzyme onto the surface layer. Optionally, more than one layer of casting may be applied to the surface layer to ensure a uniform application of enzyme.

The enzyme to be linked to the surface layer may be selected based on its specificity for a target substrate and the production of redox species that produce a detectable change in the amperometric response of the electrode.

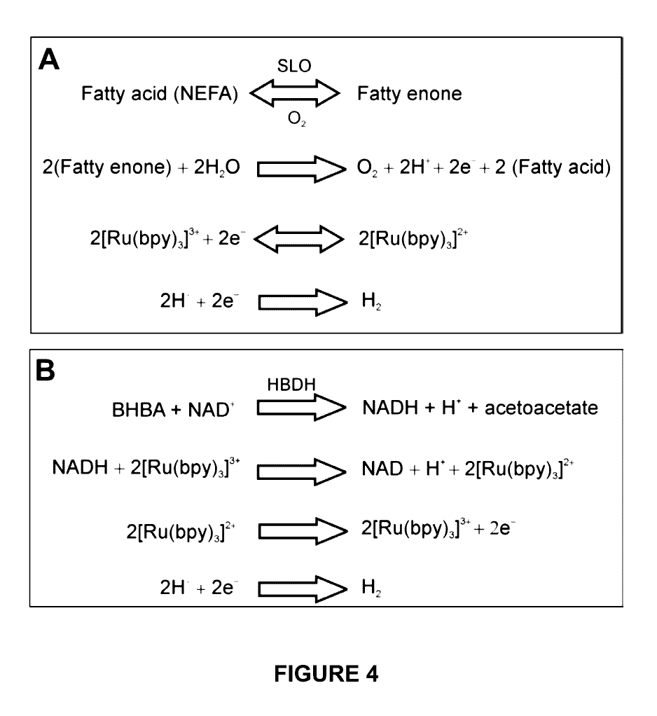

For example, in one embodiment the target substrate is non-esterified fatty acids (NEFA) and the enzyme has lipoxygenase activity. Lipoxygenases are well-known iron-containing enzymes that catalyze the oxidation of polyunsaturated fatty acids to form a peroxide of the acid. Soybean lipoxygenase-1 (SLO) has been shown to mediate the oxygenation of monounsaturated fatty acids to enones (Clapp et al., 2001, 2006). Here, SLO has been used to modify [Ru(bpy)₃]²⁺-GO electrodes for direct electrochemical oxidation of NEFA. Without being limited by theory, FIG. 4A shows the proposed reaction mechanism behind the SLO in situ electrochemical oxidation of NEFA on the surface of [Ru(bpy)₃]²⁺-GO electrode. Furthermore, lipoxygenase modified [Ru(bpy)₃]²⁺-GO electrodes have been demonstrated to be sensitive to various concentrations of standard NEFA solutions and serum samples.

In another embodiment the target substrate is beta-hydroxybutyrate and the enzyme has β-hydroxybutyrate dehydrogenase activity. In one embodiment, the surface of the electrode comprises an enzyme with β-hydroxybutyrate dehydrogenase activity and NAD+. In one embodiment, NAD+ may be was first linked to [Ru(bpy)₃]²⁺-GO using an EDC (N-(3-dimethylaminopropyl)-N′-ethylcarbodiimide) and NHS (N-hydroxysuccinimide) coupling reaction. This procedure not only minimizes the fabrication steps but also retains the durable catalytic activity of cofactor NAD+. Afterwards, HBDH enzyme may be linked onto the [Ru(bpy)₃]²⁺-GO/NAD+ electrode surface. Without being limited by theory, FIG. 4B shows the proposed reaction mechanism toward BHBA, oxidation of NADH and [Ru(bpy)₃]²⁺ at the electrode surface. Furthermore, HBDH modified [Ru(bpy)₃]²⁺-GO/NAD+ electrodes have been demonstrated to be sensitive to various concentrations of standard β-hydroxybutyrate solutions and serum samples.

In a preferred embodiment, the biosensors described herein may contain two or more electrodes that have been modified to have a surface layer of tris-ruthenium bipyridine2+ complex modified graphene oxide and have different enzymes linked to the surface layers of the two or more electrodes. For example, in one embodiment, there is provided a biosensor comprising:

a first electrode comprising a surface layer of tris-ruthenium bipyridine2+ complex modified graphene oxide and a first enzyme linked to the surface layer of the first electrode, and

a second electrode comprising a surface layer of tris-ruthenium bipyridine2+ complex modified graphene oxide and a second enzyme linked to the surface layer of the second electrode.

In one embodiment, the first electrode and second electrode are on a single substrate. Optionally, the first electrode and second electrode may be on different substrates. In one embodiment, the biosensor is for the detection of both NEFA and β-hydroxybutyrate and the biosensor comprises a first electrode with lipoxygenase enzyme linked to the surface of the first electrode and a second electrode with β-hydroxybutyrate dehydrogenase and NAD+ linked to the surface of the second electrode.

In one embodiment, the biosensors described herein comprise at least one counter electrode. Optionally, in one embodiment the biosensors described herein include at least one reference electrode. In one embodiment, the reference electrode comprises Ag/AgCl. Optionally, the counter electrode and/or reference electrode are on the same substrate or different substrates as the working electrode. In one embodiment the working electrode is an electrode comprising a surface layer of tris-ruthenium bipyridine2+ complex modified graphene oxide as described herein, optionally wherein one or more enzymes have been linked to the surface layer. In one embodiment, the biosensor does not require a reference electrode for the detection and/or quantification of a target substrate.

It will be appreciated by a person skilled in the art that standard means for applying potential and measuring current can be used for the biosensors described herein. In an embodiment, the biosensor is used in a configuration that comprises a connection of a power supply, electrodes, a sample cell and ammeter.

In one embodiment, the biosensor further comprises a microchannel for entry and exit of a liquid sample. In one embodiment, the microchannel is in fluid contact with at least a portion of the surface of a working electrode, such as a [Ru(bpy)₃]²⁺-GO electrode as described herein, and a counter electrode.

### II. Methods

In another aspect, the disclosure provides a method for detecting and/or quantifying a level of a target biomarker in a sample that use a biosensor with an electrode comprising a surface layer of tris-ruthenium bipyridine2+ complex modified graphene oxide as described herein. In one embodiment, the biomarker is a substrate for an enzyme that produces one or more redox active species upon contact with the substrate.

In one embodiment, there is provided a method for quantifying a level of a target substrate in a sample, the method comprising:

a) applying at least one voltage across the electrode of a biosensor as described herein and a counter electrode in the presence of the sample, and

b) detecting at least one current value in response to the at least one voltage.

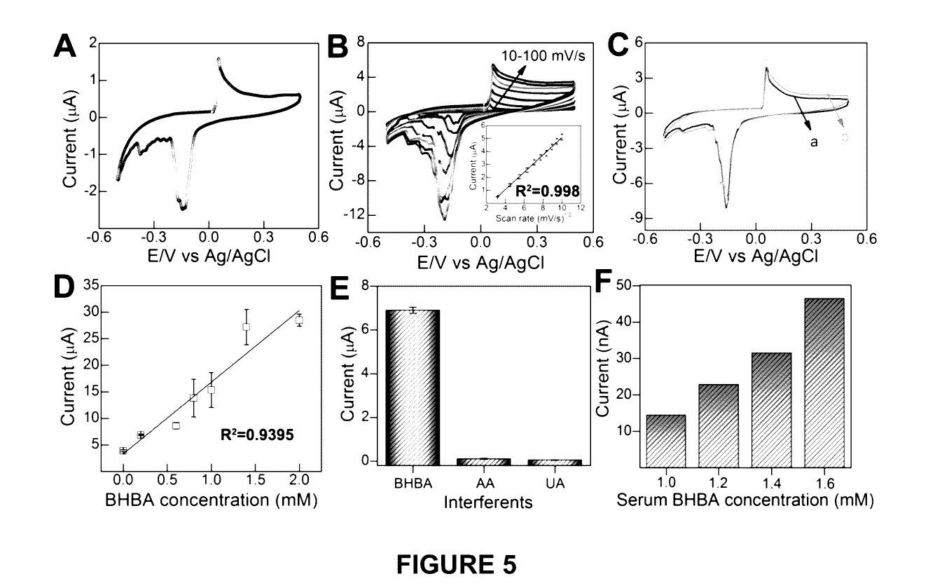

In one embodiment, the magnitude of the current value is proportional to the level of the target substrate in the sample. As shown in FIGS. 3 and 5, the use of an enzyme modified electrode as described herein exhibited a linear relationship between amperometric current and the concentration of enzyme substrates such as NEFA or BHBA.

In one embodiment, the current value may be compared to at least one standard current value, wherein the standard current value is representative of the current for a known concentration of the target substrate in the sample. In one embodiment, a standard current value me be predetermined correlation or calibration for a given target substrate and sample type. For example, in one embodiment the correlation or calibration is represented using a graph or a table or is contained in a database. In another embodiment, the correlation or calibration is encoded into software utilized by the biosensor.

Various methods known in the art of electrochemistry may be used to apply voltage to the electrode and counter electrode of a biosensor as described herein and to detect a current value. The voltage applied across the electrodes is any suitable voltage. It will be appreciated by a person skilled in the art that the voltage applied across the electrodes will depend, for example, on the resistance and the components and dimensions used for a particular biosensor. In one embodiment, the methods described herein include the use of voltammetry or cyclic voltammetry. Alternatively or in addition, the methods described herein may include the use of differential multi pulse voltammetry, double potential pulse techniques or additive differential pulse voltammetry.

In one embodiment, the methods described herein include detecting at least one current value. In one embodiment, the current value is an anodic peak current.

The sample may be any fluid sample that contains or is thought to contain a target analyte. In one embodiment, the sample is a biological fluid from a subject. In one embodiment, the biological fluid is blood, blood plasma or milk.

In one embodiment, the subject is an animal, optionally livestock or a food animal. In one embodiment the animal is a milk-producing animal. In a preferred embodiment, the animal is a bovid, optionally a dairy cow.

The biosensors and methods described herein may be used for the diagnosis of metabolic disease. For example, elevated concentrations of non-esterified fatty acids (NEFA) and β-hydroxybutyrate (BHBA) in biological fluids have been recognized as critical biomarkers for early diagnosis of dairy cow metabolic diseases.

Accordingly, in one embodiment, the biosensors and methods described herein may be used for quantifying a level of NEFA and/or BHBA in a sample from a subject for the diagnosis of metabolic disease. In one embodiment, the metabolic disease is negative energy balance (NEB). In one embodiment, the metabolic disease is clinical or subclinical ketosis (SCK).

In one embodiment, the threshold blood NEFA concentrations in dairy cows associated with SCK at pre-partum and post-partum are ≧0.3 and ≧0.6 mEq/L, respectively, whereas the BHBA concentration ranges from 1.2 to 2.9 mM (Ospina et al., 2010a).

In one embodiment, the methods described herein comprise determining a level of NEFA and/or BHBA in a test sample from a subject and identifying the subject as having a metabolic disease if the level of NEFA and/or BHBA is above a threshold concentration associated with the metabolic disease in a standard sample representative of subjects with the metabolic disease. In one embodiment, the metabolic disease is subclinical ketosis.

The following non-limiting examples are illustrative of the present application:

### Examples

### Example 1

### Experimental

### Materials

Tris(2,2′-bipyridyl)dichlororuthenium(II) hexahydrate, graphite powder (<20 μm, synthetic), soybean lipoxygenase (type I-B, lyophilized powder, 50,000 units/mg) (SLO), β-hydroxybutyric acid (BHBA), nicotinamide adenine dinucleotide (NAD+), (N-(3-dimethyl aminopropyl)-N′-ethylcarbodiimide) (EDC), and N-hydroxysuccinimide (NHS), L-ascorbic acid (AA), lactic acid solution (LA), uric acid (UA), D(+)glucose (Glu), and phosphate buffered saline (PBS) were purchased from Sigma-Aldrich. Wako HR series NEFA-HR (2) containing standard NEFA solution (1 mM oleic acid) and enzymatic assay kit were purchased from Wako Diagnostics, CA, USA, β-hydroxybutyrate dehydrogenase (HBDH) was obtained from Roche Diagnostics (Laval, QC, Canada). Except clinical serum samples, all other NEFA analyses were performed using standard oleic acid solution diluted in PBS buffer. BHBA sample solutions were prepared by diluting 3-hydroxybutyric acid in PBS buffer. Other chemicals were of analytical grade and used as received without further purification. Milli-Q water (18.2 MΩ) was used for all experiments. Various clinical serum samples of dairy cows, with known concentration of NEFA and BHBA, were provided as a gift from Animal Health Laboratory, Ontario Veterinary College, University of Guelph.

Synthesis of GO and Functionalization of [Ru(Bpy)₃]²⁺ on GO Nanosheets

Aqueous brownish colloidal GO nanosheets were synthesized by harsh oxidation of graphite powder using the modified Hummers method (Hirata et al., 2004). Functionalization of [Ru(bpy)₃]²⁺ on GO nanosheets were achieved by one-step wet-chemical synthesis, through electrostatic interaction. Typically, 10 mL of aqueous GO nanosheet (1 mg/mL) was magnetically stirred (at 800 rpm) with 10 mL of ethanolic solution of Ru(bpy)₃Cl₂ (1 mg/mL) at room temperature for overnight, protected from light. The as-obtained mixture were centrifuged (at 12,000 rpm for 45 min) and washed repeatedly with anhydrous ethanol and deionized water (DI) to remove the unreacted [Ru(bpy)₃]²⁺. Isolated [Ru(bpy)₃]²⁺-GO nanosheets were dispersed in DI water for further experimentation.

Construction of SLO-Modified GO or [Ru(Bpy)₃]²⁺-GO Electrodes for NEFA Detection

A custom-designed carbon screen-printed electrode (SPE) (from Pine Research Instrumentation, NC, USA) with an area of 2 mm in diameter was used as the substrate for constructing the working electrodes. An integrated U-shaped carbon and the circular Ag/AgCl substrates were used as counter and reference electrodes, respectively. After initial washing with DI water, carbon SPE surface was modified by drop casting 4 μL of aqueous GO or [Ru(bpy)₃]²⁺-GO suspension (1 mg/mL) and allowed to evaporate at ambient temperature for 20 min. To ensure uniform coating on the working surface typically two layers of casting were performed. As-fabricated GO or [Ru(bpy)₃]²⁺-GO electrodes were then utilized for electrochemical measurements. For NEFA detection, the above electrodes were further modified by physicosorption through drop casting 5 μL of an enzyme SBLO (0.25 mg/mL in tris buffer, pH 9). Unbound enzyme on the electrode surface was removed by gently immersion in the buffer.

Preparation of NAD+-Immobilized [Ru(Bpy)₃]²⁺-GO Nanosheets

A total of 10 mg of NHS and 2 mg of EDC were added to 500 μL of aqueous [Ru(bpy)₃]²⁺-GO nanosheets (2 mg/mL) and stirred (800 rpm) for 3 h in R.T. Then the 500 μL of 30 mM NAD+ solutions were added to the above vial and underwent magnetic stirring for 20 h to ensure complete covalent reaction. The resulting colloidal mixture was separated by centrifugation at 13,000 rpm for 10 min (at 4° C.) and washed twice with tris buffer (pH 7.5). As-obtained [Ru(bpy)₃]²⁺-GO/NAD+ conjugates were stored in refrigerator while not in use.

Construction of HBDH-Modified [Ru(Bpy)₃]²⁺-GO/NAD+ Electrodes for BHBA Detection

After an initial washing procedure, the carbon SPE surface was modified by drop casting the 4 μL of aqueous [Ru(bpy)₃]²⁺-GO/NAD+ (1 mg/mL) and allowed to evaporate at ambient temperature for 20 min. To ensure uniform coating typically two layers of casting were done. For BHBA detection, electrodes were further modified with 5 μL of an enzyme HBDH (0.25 mg/mL in PBS, pH 7.4). After 20 min of incubation time at ambient temperature the unbound enzyme on the electrode surface was removed by gently immersing in the buffer.

### Instrumentation

UV-visible absorbance spectra were measured from Cary 100 UV-vis spectrophotometer (Agilent technologies). μ-Raman spectra were recorded using RENISHAW inVia Raman microscope equipped with CCD camera and a Leica microscope. Measurements were taken using an excitation wavelength of 514 nm, laser power of 10%, exposure time of 30 s and a short working distance 50× objective lens. X-ray photoelectron spectroscopy (XPS) analysis were measured on Omicron XPS spectrometer, hemispherical analyzer that employs monochromated Al Kα radiation (hv=1486.6 eV), operating at 12 kV and 300 W. Transmission electron microscope (TEM) images were obtained from FEI-Tecani G2, operated at 200 kV. Scanning electron microscope (SEM) images were obtained using FEIInspect S50 at an accelerating voltage of 15 kV. Elemental mapping was done using Oxford XMax20 silicon drift detector and Aztec software. All electrochemical measurements were performed using SP-150 potentiostat, Bio-Logic instruments.

### Results and Discussion

Characterization of GO and [Ru(Bpy)₃]²⁺-GO Nanostructures

As shown in FIG. 1(A), UV-vis spectrum of GO exhibit the π-π* band of the polyaromatic C—C at 230 nm (Veerapandian et al., 2014). Characteristic intra-ligand transition π→π*, bpy π→π* transition and metal-to-ligand charge-transfer (MLCT) bands of [Ru(bpy)₃]²⁺ is observed at 243, 285 and 450 nm, respectively (Mori et al., 2010). A shoulder peak at 420 nm is also associated to MLCT (t2g (Ru)→π* (bpy) transitions). Due to the better optical absorption of [Ru(bpy)₃]²⁺, the hybrid [Ru(bpy)₃]²⁺-GO dispersion also exhibits the abovementioned peaks. Observed notable broadness in the individual bands of [Ru(bpy)₃]²⁺ supports the possible interaction with active groups of GO and its influence in the inherent absorbance.

Carbon lattice phase of GO studied from Raman spectroscopy (FIG. 1B) shows the characteristic D- and G-bands, at 1353 cm-1 and 1596 cm-1 corresponding to A1g symmetry and E2g phonon mode respectively (Krishnamoorthy et al., 2013). In addition to intrinsic D and G-bands, [Ru(bpy)₃]²⁺-GO nanosheets exhibit a band at 1481 cm-1 attributed to C—N stretching vibrations of bipyridyl groups (Xiao et al., 2013). The intensity ratio of I(D)/I(G) for GO and [Ru(bpy)₃]²⁺ is 0.82 and 0.74, respectively, implying that the [Ru(bpy)₃]²⁺ influenced the graphitic sp2 carbon domains on GO. The average crystallite size of sp2 domains in GO and [Ru(bpy)₃]²⁺-GO calculated according to Tunistra and Koenig's equation (Tunistra and Koenig 1970) is 20.42 and 22.63, respectively.

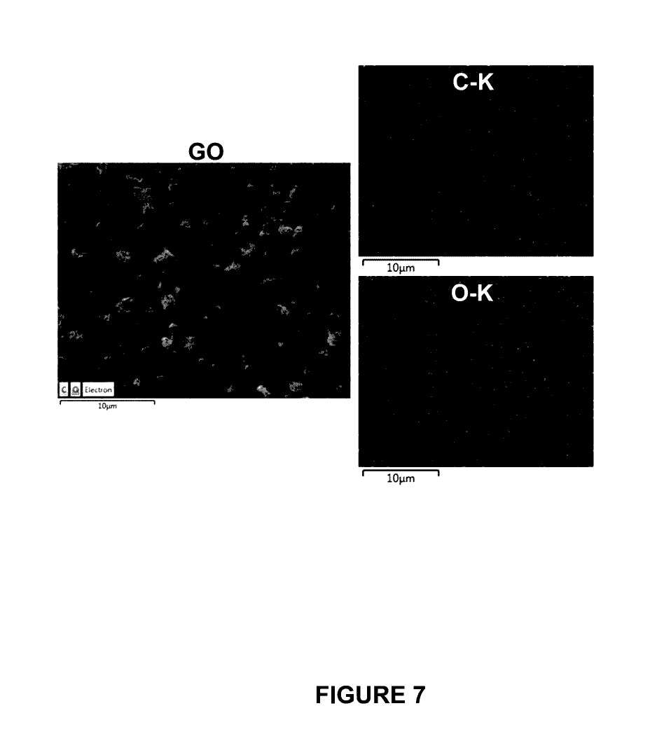

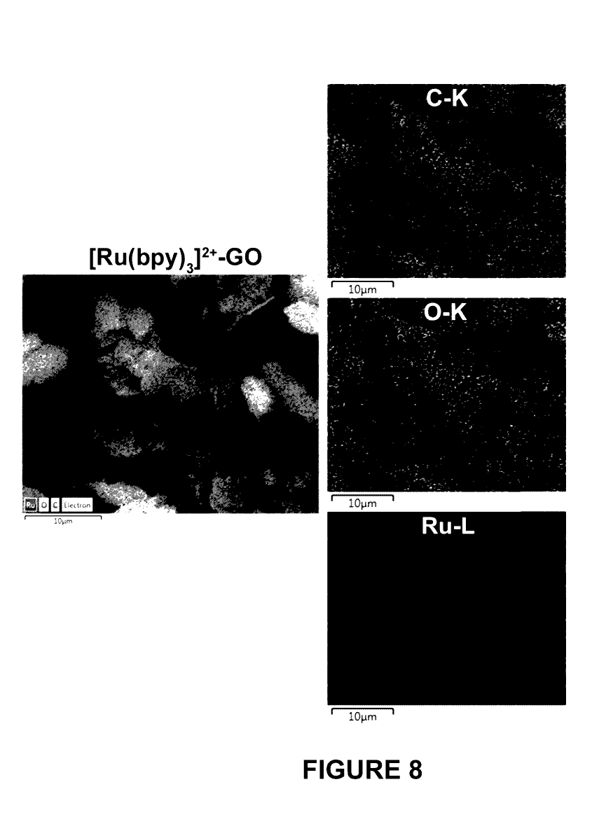

The deconvoluted Cis peaks of pristine GO are presented in FIG. 1C (i). Peaks centered at the binding energies of 288.5, 287, 286.2 and 285 eV were attributed to the oxygenated functional groups such as carboxyl, carbonyl, epoxyl and hydroxide, respectively (Koinuma et al., 2012, Krishnamoorthy et al., 2013). Peak centered at 284.2 eV is assigned to the nonoxygenated carbon lattice groups such as C═C, C—C and C—H (Krishnamoorthy et al., 2013). As shown in FIG. 1C (ii), functionalization of [Ru(bpy)₃]²⁺ significantly altered the chemical groups of GO, that include presence of new peak centered at 281.1 eV, ascribed to the Ru3d_{5/2} (Agnes et al., 2009). In addition to minor shift in the binding energies of epoxyl (from 286.2 to 285.7 eV) and hydroxyl (from 285 to 285.2 eV) groups, absence of carboxyl and carbonyl group signals indicate that the [Ru(bpy)₃]²⁺ are chemically interacted on the surface of GO. This result is further supported by the elemental mapping analysis, provided in the supporting information (FIG. 7 and FIG. 8). Few layers of ultra-thin sheets of GO and [Ru(bpy)₃]²⁺-GO observed from TEM, and microclusters of sheetlike structures with larger network studied from SEM are presented in FIGS. 1 (D) and (E), respectively.

Construction of [Ru(Bpy)₃]²⁺-GO Based Sensor Platform

Due to its multiple oxygenated functional groups, GO is generally believed to be an insulating material. Considering the cost-effective and effortless scalable synthesis, significant efforts have been made to improve the electrochemical properties of GO for use as a biosensor, including elemental doping and functionalization of hybrid inorganic/organic structures, chemical reduction and photoirradiation. Such modified GO-based materials have been demonstrated for a range of biosensors that include, homocysteine (Kannan et al., 2013), quercetin (Veerapandian et al., 2014), estriol (Cincotto et al., 2015), botulinum neurotoxin A (Chan et al., 2015), sialic acid and Listeria monocytogenes (Veerapandian et al., 2015).

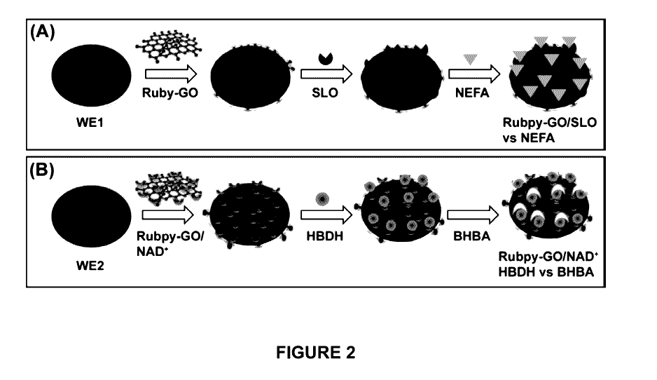

Here, there is provided a Ru(bpy)₃]²⁺-GO based sensor platform. As shown in FIG. 2, at first the working surface (2 mm in diameter) of customized SPE was modified with [Ru(bpy)₃]²⁺-GO or [Ru(bpy)₃]²⁺-GO/NAD+ through a drop casting method. Afterward, enzyme SLO and HBDH were immobilized for direct electrochemical detection of NEFA and BHBA, respectively. Due to its matrix like structure with abundant chemical groups, GO-based hybrid materials are readily adhered onto the electrode surface. Of note, using screen-printing technology the suspension of prepared nanomaterials could be conveniently scalable for mass production with high precision. Advantages of SPE include miniaturization, ease of operation, portability, reliability and modest fabrication cost.

Electrochemical Properties of [Ru(Bpy)₃]²⁺-GO Electrodes for NEFA Detection

FIG. 3A shows comparative cyclic voltammograms (CV) of bare carbon, GO and [Ru(bpy)₃]²⁺-GO nanosheets modified electrodes measured under PBS buffer (7.4) at a scan rate of 20 mV/s, without NEFA. It has been observed that bare carbon and GO-modified electrodes don't exhibit characteristic redox behavior. Interestingly, the [Ru(bpy)₃]²⁺-GO nanosheets modified carbon SPE showed a well-defined peak centered at Eₚₐ=+0.055 V and E_{pc}=−0.1 V vs Ag/AgCl, which is attributed to the redox behavior of Ru_{II}/Ru_{III}. The observed oxidation potential (+0.055 V) is comparably less positive than the previously reported, carbon paste, ITO and graphene-modified glassy carbon, electrodes (Wohnrath et al., 2005; Wohnrath et al., 2006; Xu et al., 2015). Such enhanced redox response is mainly due to the stable interaction of [Ru(bpy)₃]²⁺ into the basal and edges of GO sheets. Therefore, a significant alteration in the carbon/oxygen atomic ratio creates new sp3 and sp2 domains on the lattice network and its derived electroactive charge carriers. The scan rate dependence of the CV response from [Ru(bpy)₃]²⁺-GO electrode was examined (FIG. 3B) and found that the redox current is proportional to the scan rate (υ_{1/2}). Representative anodic peak current fit exhibit a correlation co-efficient of 0.997, suggesting the surface-confined reaction process.

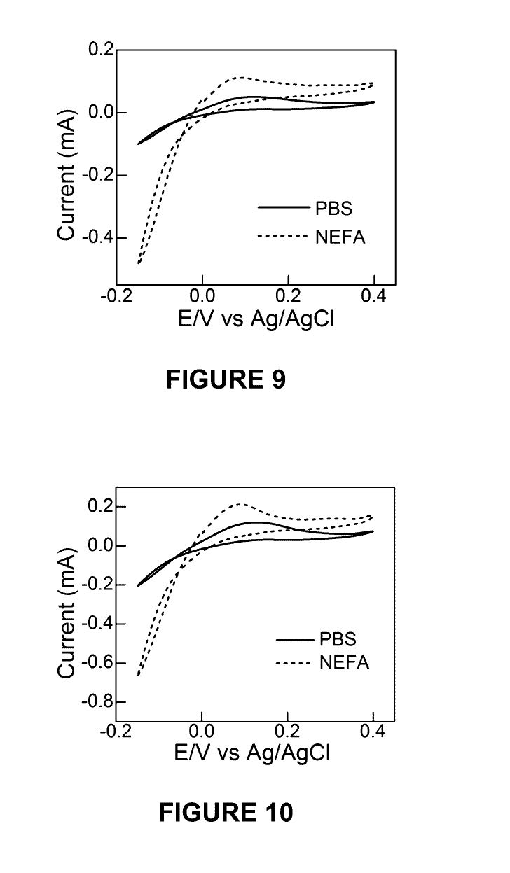

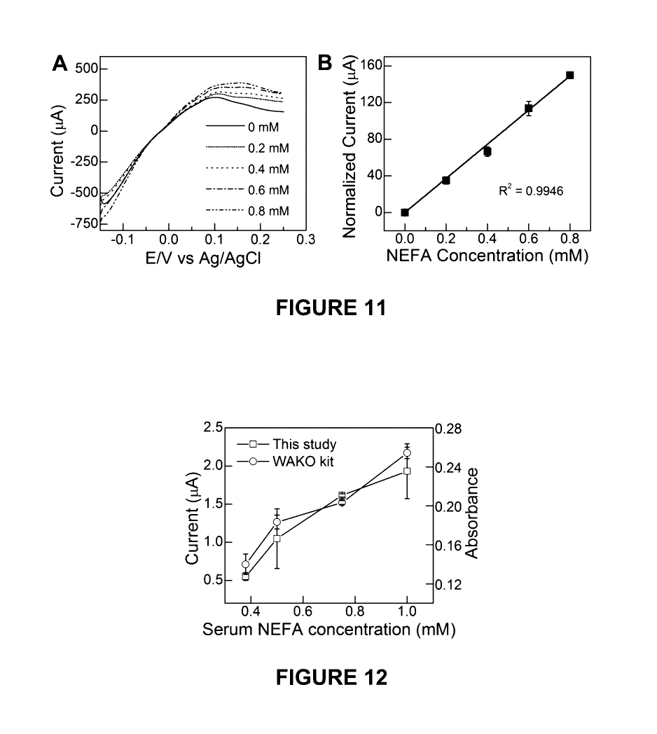

CV study of pristine SLO modified [Ru(bpy)₃]²⁺-GO electrode in PBS buffer exhibit a broad anodic peak centered at +0.11 V. Absence of an inherent cathodic peak (under these potential window) attributed to the cycle RuIII to RuII is perhaps due to the existence of SLO, which hindered the reduction reaction at the electrode interface. Upon interaction with NEFA, the anodic peak current generated from the [Ru(bpy)₃]²⁺-GO/SLO electrode is noticeably higher than the pristine one, with a minor shift in the peak potential (from +0.11 to +0.125 V). This implies that a direct electrochemical oxidation of NEFA is feasible at the SLO supported [Ru(bpy)₃]²⁺-GO electrode. CVs of SLO-modified bare carbon and GO electrodes in absence and presence of NEFA molecules were also measured and the results are provided in FIGS. 9 and 10. Complementary linear sweep voltammetric study on the [Ru(bpy)₃]²⁺-GO/SLO electrode showed a linear relationship to the NEFA concentrations with a correlation coefficient of 0.9946 (FIG. 11). Preliminary voltammetric investigations showed that the change of anodic peak current specifically at +0.17 V is found to be an optimal potential to monitor the sensing ability. Hence, an applied potential of +0.17 V was used for amperometric sensing of NEFA samples. Presence of SLO on the surface of [Ru(bpy)₃]²⁺-GO readily oxidizes the free NEFA and hence supplies electrons to the electrode. Plausible electrochemical catalytic reaction of SLO and inherent redox reaction of an electrode are explained in FIG. 4A. Amperometric response of the [Ru(bpy)₃]²⁺-GO/SLO electrodes to different concentrations of NEFA are illustrated in the FIG. 3D calibration plot, which shows a high sensitivity of 42 ρA mM-1, in the linear detection range of 0.1 to 1.0 mM. Further, specificity of the proposed electrode was evaluated in presence of various potential interferents, viz., AA, LA, UA and Glu. To ensure the specificity a three-fold increase of interferent's concentration (1.2 mM) were utilized. As observed in FIG. 3E, the amperometric response against the 0.4 mM concentration of NEFA is still better than the studied interferent's concentration, indicating that the enzyme SLO on the surface of [Ru(bpy)₃]²⁺-GO selectively oxidizes NEFA, resulting in uninterrupted electron transfer process.

Further, the practical application of the proposed electrode toward real clinical samples for monitoring NEFA was explored. At first different dairy cows suspected with NEB were selected and their respective serum samples of various NEFA concentrations (within the critical threshold) were obtained from the Animal Health Laboratory at the University of Guelph. The amperometric sensing ability of the [Ru(bpy)₃]²⁺-GO/SLO electrode for NEFA was measured with the selected serum samples (within critical threshold), such as 0.38, 0.5, 0.75 and 1.0 mM, respectively. Compared to standard NEFA samples (FIG. 3D), the current sensitivity of the electrodes with serum samples are decreased perhaps due to the presence of multiple serum components. However, as shown in FIG. 3F, the changes in individual current values and serum NEFA concentrations have a relationship suitable for potential detection application. Moreover, the results were consistent with WAKO kit values shown in FIG. 12.

Electrochemical Properties of [Ru(Bpy)₃]²⁺-GO/NAD+ Electrodes for BHBA Detection

Upon covalent immobilization of NAD+ molecules on the surface of GO and [Ru(bpy)₃]²⁺-GO, a preliminary CV measurement was done in PBS buffer (pH 7.4) at a scan rate of 20 mV/s. The pristine GO nanosheets exhibited comparatively a weak redox behavior with poor reproducibility (results not shown). However, as shown in FIG. 5A, the [Ru(bpy)₃]²⁺-GO/NAD+ electrode exhibited a well resolved anodic and cathodic peak potential at +0.06 and −0.14 V, respectively. The observed shift in the inherent anodic (from +0.055 to +0.06 V) and cathodic peak (from −0.1 to −0.14 V) potentials, attributed to the redox behavior of RuII/RuIII is perhaps due to the presence of NAD+. This result is further supported by the observed moderate cathodic peak centered at −0.35 V, which is ascribed to the reduction of NAD+ to NADH. The reduction of coenzyme NAD+ varies, depending on the type of electrode and active materials immobilized on the surface of electrode. For instance, the NAD+ reduction potential on the pristine glassy carbon electrode and ruthenium nanoparticles-modified glassy carbon electrode in PBS was reported to be −1.2 and −1.18 V, respectively (Rahman et al., 2011). A decrease in the reduction potential of NAD+ at −0.65 V was reported in a study of SWCNT functionalized on graphite electrode (Khorsand et al., 2013). The decreased reduction potential observed for the coenzyme in the present study is probably due to the distinct electroactive surface chemistry of [Ru(bpy)₃]²⁺-GO nanosheets. The scan-rate dependent CV study further revealed that the observed redox peak potentials are in relationship. Representative anodic peak current plot against the square root of scan rate exhibit a correlation coefficient of 0.998, indicating the surface-confined process suitable for biosensor studies.

CV study on [Ru(bpy)₃]²⁺-GO/NAD+/HBDH electrode in the absence of BHBA illustrated in FIG. 5C(a) exhibit the anodic and cathodic peak potential at +0.06 and −0.16 V, respectively. Upon interaction with BHBA, the anodic peak current attributed to oxidation of RuII is noticeably increased, whereas the reduction peak current attributed to RuIII is decreased, this perhaps due to the multiple enzymatic reactions at the interface such as HBDH mediated dehydrogenation of BHBA to acetoacetate and the catalytic activity of cofactor NAD+ to NADH. The moderate cathodic peak at −0.35 V responsible for reduction of NAD+ is broadened (in comparison to FIG. 5A) and also increased after interacting with BHBA, supporting the cascade of reaction at the electrode proposed in FIG. 4B. Based on these preliminary CV results, the anodic peak potential at +0.06 V appears suitable for amperometric sensing of BHBA. FIG. 5D represents the calibration curve fit obtained from the amperometric experiments on standard BHBA samples in PBS buffer, it was found that the [Ru(bpy)₃]²⁺-GO/NAD+/HBDH electrode exhibited a linear amperometric response as a function of BHBA concentration with a correlation coefficient of 0.9395. The current sensitivity of the proposed BHBA biosensor measured (as the slope of the plot) in the present detection range is determined to be 22±2.51 ρA mM-1.

Ensuring the selectivity and specificity of the new biosensor is important for successful applications in the field. UA and AA are common interferents that are evaluated for electrochemical BHBA sensors. FIG. 5E shows the amperometric response of the proposed sensor against 0.2 mM of BHBA and a six-fold increase of interferent concentration (1.2 mM). Due to its well-known enzymatic activity, HBDH modified on the electrode surface exhibits an amplified current response toward the BHBA than the studied interferents. The negligible current observed for the interferents might be attributed to the non-specific adsorption of molecules onto the biosensor surface. In order to evaluate the practical sensing ability of the [Ru(bpy)₃]²⁺-GO/NAD+/HBDH electrode in biological fluids, serum samples from dairy cows with clinical ketosis are utilized. Particularly serum samples with a clinical cut-off value concentration between 1 to 1.6 mM of BHBA are selected for the analysis. FIG. 5F depicts the amperometric response observed from different electrodes studied with serum samples. Compared to standard BHBA samples diluted in buffer, the current response during the real serum analysis are lower, however the individual electrodes are still able to deliver a concentration dependent amperometric signal. Further optimization especially on the electrode surface treatment and enzyme immobilization may improve the current sensitivity and enhanced stability of the BHBA sensor.

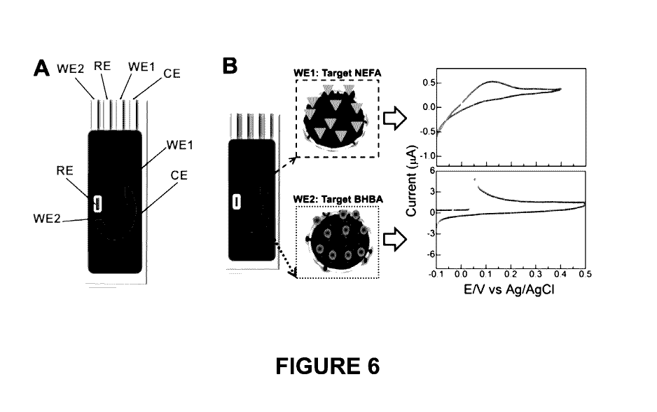

The electrochemical approach described herein has been shown to be fast (<1 min) and efficient in comparison with conventional assays. Further, using a dual screen-printed electrode system the sensing mechanism can be effectively integrated and feasible for the rapid assay of two or more biomarkers such as NEFA and BHBA. Such integration would not only reduce the cost of individual assays but also improve the standard for early diagnosis of metabolic diseases and provide point-of-care monitoring. FIG. 6 illustrates the dual electrode design and model voltammetric signal for monitoring NEFA and BHBA.

A field deployable biosensor platform based on [Ru(bpy)₃]²⁺-GO nanosheet for the simultaneous electrochemical detection of NEFA and BHBA has been demonstrated. Immobilization of lipoxygenase on the electrode surface selectively catalyzes the NEFA into fatty enones and influences the inherent redox reaction at the interface resulting in a concentration dependent amperometric response. The covalently functionalized [Ru(bpy)₃]²⁺-GO/NAD+ electrode supports the catalytic activity of HBDH, which selectively converts BHBA into acetoacetate, in association with inherent redox reaction of NAD+/NADH, thereby electrochemically determines the concentration of BHBA. Both electrode systems possess high specificity and show excellent linear dependence toward various concentrations of the standard NEFA/BHBA as well as serum samples. The enzymatic amperometric biosensor is relatively simple and rapid and does not require sample pre-treatment. The dual sensing approach and proposed electrode design based on two working electrodes with common reference and counter electrodes in a single chip may be especially useful for on-farm point-of-care diagnostics and for the diagnosis of negative energy balance and/or clinical or subclinical ketosis.

All publications, patents and patent applications are herein incorporated by reference in their entirety to the same extent as if each individual publication, patent or patent application was specifically and individually indicated to be incorporated by reference in its entirety.

### REFERENCES

Agnes, C., Arnault, J. C., Omnes, F., Jousselme, B., Billon, M., Bidan, G., Mailley, P., 2009. XPS study of ruthenium tris-bipyridine electrografted from diazonium salt derivative on microcrystalline boron doped diamond. Phys. Chem. Chem. Phys., 11, 11647-11654.
Antaliková, J., Simon, M., Jankovicová, J., Horovská, L. U., Fábryová, K., & Hluchy, S. (2007). Biochemical and Histochemical Characterization of the Cattle V Red Blood Cell Antigen with Monoclonal Antibody IVA-41. Hybridoma, 26(4), 255-258.
Chan, C. Y., Guo, J., Sun, C., Tsang, M. K., Tian, F., Hao, J., Chen, S., Yang, M., 2015. A reduced graphene oxide-Au based electrochemical biosensor forultrasensitive detection of enzymatic activity of botulinum neurotoxin A. Sens. Actuat. B 220, 131-137.
Cincotto, F. H., Canevari, T. C., Machado, S. A. S., Sánchez, A., Barrio, M. A. R., Villalonga, R., Pingarrón, J. M., 2015. Reduced graphene oxide-Sb2O5 hybrid nanomaterial for the design of a laccase-based amperometric biosensor for estriol. Electrochim. Acta 174, 332-339.
Clapp, C. H., Senchak, S. E., Stover, T. J., Potter, T. C., Findeis, P. M., Novak, M. J., 2001. Soybean lipoxygenase-mediated oxygenation of monounsaturated fatty acids to enones. J. Am. Chem. Soc. 123, 747-748.
Clapp, C. H., Strulson, M., Rodriguez, P. C., Lo, R., Novak, M. J., 2006. Oxygenation of monounsaturated fatty acids by soybean lipoxygenase-1: evidence for transient hydroperoxide formation. Biochemistry 45, 15884-15892.
Fang, L., Wang, S-H., Liu, C-C., 2008. An electrochemical biosensor of the ketone 3-[beta]hydroxybutyrate for potential diabetic patient management. Sens. Actuat. B Chem, 129,818-825.
Forrow, N. J., Sanghera, G. S., Wafters, S. J., Watkin, J. L., 2005. Development of a commercial amperometric biosensor electrode for the ketone D-3-hydroxybutyrate. Biosens. Bioelectron. 20, 1617-1625.
Garverick, H. A., Harris, M. N., Vogel-Bluel, R., Sampson, J. D., Bader, J., Lamberson, W. R., Spain, J. N., Lucy, M. C., Youngquist, R. S., 2013. Concentration of nonesterified fatty acids and glucose in blood of periparturient dairy cows are indicative of pregnancy success at first insemination. J. Dairy Sci. 96, 181-188.
Hirata M, Gotou T, Horiuchi S, Horiuchi, S., Fujiwara, M., Ohba, M., 2004. Thin-film particles of graphite oxide 1: High-yield synthesis and flexibility of the particles. Carbon 42, 2929-2937.
Jorjong, S., van Knegsel, A. T. M., Verwaeren, J., Val Lahoz, M., Bruckmaier, R. M., DeBaets, B., Kemp, B., Fievez, V., 2014. Milk fatty acids as possible biomarkers to early diagnose elevated concentrations of blood plasma nonesterified fatty acids in dairy cows. J. Dairy Sci. 97, 7054-7064.
Kannan, P., Maiyalagan, T., Sahoo, N. G., Opallo, M., 2013. Nitrogen doped grapheme nanosheet supported platinum nanoparticles as high performance electrochemical homocysteine biosensors. J. Mater. Chem. B 1, 4655-4666.
Kang, J., Hussain, A. T., Catt, M., Trenell, M., Haggett, B., Yu, E. H., 2014. Electrochemical detection of non-esterified fatty acid by layer-by-layer assembled enzyme electrodes. Sens. Actuat. B-Chem, 190, 535-541.
Khorsand, F., Azizi, M. D., Naeemy, A., Larijani, B., & Omidfar, K. (2013). An electrochemical biosensor for 3-hydroxybutyrate detection based on screen-printed electrode modified by coenzyme functionalized carbon nanotubes. Molecular biology reports, 40(3), 2327-2334.
Koinuma, M., Ogata, C., Kamei, Y., Hatakeyama, K., Tateishi, H., Watanabe, Y., Taniguchi, T., Gezuhara, K., Hayami, S., Funatsu, A., Sakata, M., Kuwahara, Y., Kurihara, S., Matsumoto, Y., 2012. Photochemical Engineering of Graphene Oxide Nanosheets, J. Phys. Chem. C 116, 19822-19827.
Krishnamoorthy, K., Veerapandian, M., Yun, K. S., Kim, S. J., 2013. The chemical and structural analysis of graphene oxide with different degrees of oxidation. Carbon 53,38-49.
Kwan, R. C. H., Hon, P. Y. T., Mak, W. C., Law, L. Y., Hu, J., Renneberg, R., 2006. Biosensor for rapid determination of 3-hydroxybutyrate using bienzyme system. Biosens. Bioelectron. 21, 1101-1106.
Li, G., Ma, N. Z., Wang, Y., 2005. A new handheld biosensor for monitoring blood ketones. Sens Actuat. B Chem. 109, 285-290.
Miksa, I. R., Buckley, C. L., Poppenga, R. H., 2004, Detection of nonesterified (free) fatty acids in bovine serum: comparative evaluation of two methods. J. Vet. Diagn. Invest. 16, 139-144.
Mori, K., Kawashima, M., Cheand, M., Yamashita, H., 2010. Enhancement of the photoinduced oxidation activity of a ruthenium(II) complex anchored on silica-coated silver nanoparticles by localized surface plasmon resonance. Angew. Chem., Int. Ed., 49, 8598-8601.
Nielen, M., Aarts, M. G. A., Jonkers, Ad G. M., Wensing, T., Schukken, Y. H., 1994. Evaluation of two cowside tests for the detection of subclinical ketosis in dairy cows. Can. Vet. J, 35, 229-232.
Nishikiori, M., Iizuka, H., Ichiba, H., Sadamoto, K., Fukushima, T., 2014. Determination of Free Fatty Acids in Human Serum by HPLC with Fluorescence Detection. J. Chromatogr. Sci. 53, 537-541.
Ospina, P. A., Nydam, D. V., Stokol, T., Overton, T. R., 2010a. Evaluation of nonesterified fatty acids and/β-hydroxybutyrate in transition dairy cattle in the northeastern United States: Critical thresholds for prediction of clinical diseases. J. Dairy Sci. 93, 546-554.
Ospina, P. A., Nydam, D. V., Stokol, T., Overton, T. R., 2010b. Associations of elevated nonesterified fatty acids and/β-hydroxybutyrate concentrations with early lactation reproductive performance and milk production in transition dairy cattle in the northeastern United States. J. Dairy Sci. 93, 1596-1603.
Rahman, G., Lim, J. Y., Jung, K. D., Joo, O.-S., 2011. Electrodeposited Ru Nanoparticles for Electrochemical Reduction of NAD+ to NADH. Int. J. Electrochem. Sci. 6, 2789-2797.
Roche, https://e-labdoc.roche.com/LFR PublicDocs/ras/11383175001 en 09.pdf retrieved on 13/05/2015
Tuinstra, F.; Koenig, J. L. 1970. Raman spectrum of graphite. J. Chem. Phys. 53, 1126-1130.
Veerapandian, M., Seo, Y. T., Yun, K. S., Lee, M.-H., 2014. Graphene oxide functionalized with silver@silica-polyethylene glycol hybrid nanoparticles for direct electrochemical detection of quercetin. 2014. Biosens. Bioelectron. 58, 200-204.
Veerapandian, M., Neethirajan, S., 2015. Graphene oxide chemically decorated with Ag-Ru/chitosan nanoparticles: fabrication, electrode processing and immunosensing property. RSC Adv. DOI: 10.1039/C5RA15329H.
Wako, http://www.wakodiagnostics.com/r_nefa.html pdf retrieved on 13/05/2015
Wohnrath, K., Pessoa, C. A., dos Santos, P. M., Garcia, J. R., Batista, A. A., Oliveira Jr, O. N., 2005. Electrochemical properties of a ruthenium complex immobilized as thin films and in carbon paste electrodes. Prog. Solid State Chem. 33, 243-252.
Wohnrath, K., dos Santos, P. M., Sandrino, B., Garcia, J. R., Batista, A. A., Oliveira Jr, O. N., 2006. A novel binuclear ruthenium complex: spectroscopic and electrochemical characterization, and formation of Langmuir and Langmuir-Blodgett films. J. Braz. Chem. Soc., 17, 1634-1641.
Xiao, F. N., Wang, M., Wang, F. B., Xia, X. H., 2014. Graphene-ruthenium(II) complex composites for sensitive ecl immunosensors. Small 10, 706-716.
Xiao, B., Wang, X., Huang, H., Zhu, M., Yang, P., Wang, Y., Du, Y., 2013, Improved superiority by covalently binding dye to graphene for hydrogen evolution from water under visible-light irradiation. J. Phys. Chem. C 117, 21303-21311.
Xu, Y., Cao, M., Liu, H., Zong, X., Kong, N., Zhang, J., Liu, J., 2015. Electrontransfer study on graphene modified glassy carbon substrate via electrochemical reduction and the application for tris(2,2′-bipyridyl) ruthenium(II) electrochemiluminescence sensor fabrication. Talanta 139, 6-12.
Yang, M., Fujino, T., 2014. Quantitative analysis of free fatty acids in human serum using biexciton auger recombination in cadmium telluride nano particles loaded on zeolite. Anal. Chem. 86, 9563-9569.
Zhang, Y., Qiu, L., Wang, Y., Qin, X., Li, Z., 2014. High-throughput and high-sensitivity quantitative analysis of serum unsaturated fatty acids by chip based nanoelectrospray ionization-Fourier transform ion cyclotron resonance mass spectrometry: Early stage diagnostic biomarkers of pancreatic cancer. Analyst 139, 1697-1706.

1. An electrochemical biosensor comprising:
an electrode comprising a surface layer of tris-ruthenium bipyridine²⁺ complex modified graphene oxide; and
at least one enzyme linked to the surface layer.

2. The biosensor of claim 1, wherein the electrode comprises carbon, gold, silver, copper, aluminum, graphite, brass, platinum, palladium or titanium.

3. The biosensor of claim 2, wherein the electrode is a screen printed electrode.

4. The biosensor of claim 1, wherein the tris-ruthenium bipyridine²⁺ complex modified graphene oxide is drop cast onto the surface of the electrode.

5. The biosensor of claim 1, wherein the surface layer of ruthenium bipyridine complex modified graphene oxide has a sheetlike structure.

6. The biosensor of claim 1, wherein the enzyme is covalently linked to the surface layer of tris-ruthenium bipyridine²⁺ complex modified graphene oxide.

7. The biosensor of claim 1, wherein the at least one enzyme is lipoxygenase or the at least one enzyme is β-hydroxybutyrate dehydrogenase and the electrode further comprises nicotinamide adenine dinucleotide (NAD+) linked to the surface layer.

8. (canceled)

9. (canceled)

10. The biosensor of claim 1, comprising:
a first electrode comprising a surface layer of tris-ruthenium bipyridine²⁺ complex modified graphene oxide and a first enzyme linked to the surface layer of the first electrode, and
a second electrode comprising a surface layer of tris-ruthenium bipyridine²⁺ complex modified graphene oxide and a second enzyme linked to the surface layer of the second electrode,
wherein the first electrode and second electrode are on a single substrate.

11. The biosensor of claim 10, wherein the first enzyme is lipoxygenase and the second enzyme is β-hydroxybutyrate dehydrogenase.

12. The biosensor of claim 1, further comprising at least one counter electrode and optionally at least one reference electrode.

13. (canceled)

14. The biosensor of claim 1, wherein the biosensor is in a hand-held system for detecting a target substrate in a fluid sample or an in-line system for detecting a target substrate in a fluid sample.

15. (canceled)

16. A method for quantifying a level of a target substrate in a sample, the method comprising:
a) applying at least one voltage across the electrode of a biosensor as defined in claim 1 and a counter electrode in the presence of the sample, and
b) detecting at least one current value in response to the at least one voltage,
wherein the magnitude of the current value is proportional to the level of the target substrate in the sample.

17. The method of claim 16, further comprising comparing the at least one current value to at least one standard current value, wherein the standard current value is representative of the current for a known concentration of the target substrate in the sample.

18. The method of claim 16, wherein steps a) and b) comprise voltammetry, optionally cyclic voltammetry, differential multi pulse voltammetry, double potential pulse techniques or additive differential pulse voltammetry.

19. The method of claim 16, wherein detecting at least one current value comprises detecting an anodic peak current.

20. The method of claim 16, wherein the target substrate is non-esterified fatty acids (NEFA) and the biosensor comprises lipoxygenase linked to the surface layer.

21. The method of claim 16, wherein the target substrate is β-hydroxybutyrate (BHBA) and the biosensor comprises β-hydroxybutyrate dehydrogenase and NAD+ linked to the surface layer.

22. The method of claim 16, wherein the sample is a biological fluid from a subject and the biological fluid is blood, blood plasma or milk.

23. (canceled)

24. (canceled)

25. The method of claim 16 for the detection of metabolic disease.

26. The method of claim 25, wherein the metabolic disease is negative energy balance (NEB) or clinical or subclinical ketosis.

27. (canceled)
